(19) United States
(12) Patent Application Publication
(10) Pub. No.: US 2010/0137987 A1
(43) Pub. Date: Jun. 3, 2010

(54) Intramedullary Repair System for Vertebra Fractures

(75) Inventors: Edward Diao, San Francisco, CA (US); Harold F. Carrison, Pleasanton, CA (US); Samuel Wade Johnson, Littleton, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: EndoSpinal, Inc., San Francisco, CA (US)

(21) Appl. No.: 12/622,320

(22) Filed: Nov. 19, 2009

Related U.S. Application Data
(60) Provisional application No. 61/116,074, filed on Nov. 19, 2008.

(51) Int. Cl. A61F 2/44

(52) U.S. Cl. 623/17.15; 623/17.11

(57) ABSTRACT

An system for reducing a fracture in an vertebral body is disclosed herein. The system may include incremental implant elements and a delivery device. The incremental implant elements are configured for percutaneous delivery into, and accumulation within, a volume of the vertebral body. The delivery device is configured to percutaneously deliver the incremental implant elements into the volume of the vertebra body in an incremental fashion. As the incremental implant elements are incrementally delivered into the volume of the vertebra body, the accumulation of the incremental members in the volume of the vertebra body places the fracture in the vertebra body in a reduced state.

### CROSS REFERENCE TO RELATED APPLICATIONS

The present application claims priority under 35 U.S.C. §119 to U.S. provisional patent application 61/116,074, which was filed Nov. 19, 2008 and is hereby incorporated by reference in its entirety into the present application.

The present application is also related to co-pending U.S. patent application Ser. No. 12/______, which was filed on Nov. 19, 2009 and entitled “Intramedullary Repair System For Bone Fractures” and is hereby incorporated by reference in its entirety into the present application.

### FIELD OF THE INVENTION

The present invention relates to orthopedic apparatus and methods. More specifically, the present invention relates to intramedullary vertebra fracture repair devices and methods.

### BACKGROUND OF THE INVENTION

Successfully treating vertebra fractures has been difficult, at best. For example, it is not uncommon for the vertebra fracture to fail to remain completely reduced during the surgical procedure and post operative healing, necessitating revisions. The treated vertebra fracture can be less strong than desired. Also, patient soft tissue trauma can be significant, increasing patient pain and the period needed for healing. Finally, surgical procedure times can be significant.

There is a need in the art for devices and methods that offer improved outcomes for the treatment of vertebra fractures, resulting in better aligned and stronger healed fractures, reducing the likelihood of a revision being necessary, and reducing the damage to soft tissue adjacent the fracture. There is also a need in the art for devices and methods that offer a reduction in the surgical time required for the treatment of bone fractures.

### BRIEF SUMMARY OF THE INVENTION

An system for reducing a fracture in an vertebral body is disclosed herein. In one embodiment, the system includes incremental implant elements and a delivery device. The incremental implant elements are configured for percutaneous delivery into, and accumulation within, a volume of the vertebral body. The delivery device is configured to percutaneously deliver the incremental implant elements into the volume of the vertebra body in an incremental fashion.

An intramedullary implant for reducing a fracture in an vertebral body is disclosed herein. In one embodiment the implant includes generally opposed plates coupled together and configured to deploy from a percutaneously deliverable configuration to a deployed configuration wherein the plates are displaced from each other.

A method of reducing a fracture in a vertebra is disclosed herein. In one embodiment, the method includes: percutaneously introducing an implant into a volume of the vertebra body; causing first and second members of the implant to displace away from each other within the volume, thereby placing the fracture in the vertebra body in a reduced state; and fixing the first and second members relative to each other so as to maintain the fracture of the vertebra body in the reduced state.

Another method of reducing a fracture in a vertebra is also disclosed herein. In one embodiment, the method includes: percutaneously introducing incremental implant members into a volume of the vertebra body, the accumulation of the incremental members in the volume of the vertebra body placing the fracture in the vertebra body in a reduced state.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIGS. 1A and 1B are side isometric views of the implant in a deployed state and in a collapsed or delivery state, respectively.

FIGS. 2A ad 2B are side views of an implant similar to the implant depicted in FIGS. 1A and 1B, the implant depicted in a deployed state and in a collapsed or delivery state, respectively.

FIGS. 3A and 3B are plan views of the coupling side of a plate of the implant and a bone engagement side of the plate, respectively.

FIGS. 4A and 4B are, respectively, a side isometric view of the implant exiting a delivery tubular body (e.g., trocar, introducer sheath, needle, etc.) and a transverse cross sectional view of the tubular body and implant as taken along section line 4B-4B in FIG. 4A.

FIGS. 5A and 5B are side views of the implant in a deployed state and in a collapsed or delivery state, respectively, wherein the implant jack includes a single leg.

FIGS. 6A-6B, which are side views of the implant in a partially deployed state and a maximum deployed state, respectively, wherein the implant jack includes a locking mechanism.

FIG. 7 is a side view of the implant partially deployed, wherein the implant jack includes a ratchet mechanism.

FIGS. 8A and 8B are side isometric views of the implant in a collapsed or delivery state and a deployed state.

FIGS. 9A-9F are posterior-anterior cross sectional elevations of a vertebra having a fracture at a anterior portion of the vertebral body, wherein FIGS. 9A-9F depict the progression of the process of reducing the fracture with the implant of FIGS. 2A-2B.

FIGS. 10A and 10B, which are the same views as FIGS. 9A and 9B, except depicting the embodiment of FIGS. 8A and 8B being deployed.

FIG. 11, which is a side elevation view (in partial cross section) of a delivery device that incrementally delivers incremental implant elements that accumulate to form the implant.

FIGS. 12A-12E are posterior-anterior cross sectional elevations of a vertebra having a fracture at a anterior portion of the vertebral body, wherein FIGS. 12A-12E depict the progression of the process of reducing the fracture with the accumulated or built up implant.

FIGS. 13A and 13B are the same views of the fully reduced vertebral body as FIG. 12E, except material is being injected into the volume of the vertebral body.

FIGS. 14A and 14B are side view of the helical spring implant elements in a constrained state and in a free state, respectively.

FIGS. 15A and 15B are the same views as FIGS. 12A and 12B, except of the helical spring implant elements of FIGS. 14A and 14B being deployed.

FIGS. 16A and 16B are, respectively, a side view and an end view of the cylindrical implant elements.

FIGS. 17A and 17B are the same views as FIGS. 12A and 12B, except of the cylindrical implant elements of FIGS. 16A and 16B being deployed.

FIG. 18 is a side view of an accumulated or built-up implant employing shaped mating/interlocking implant elements and cylindrical implant elements that transconfigure into planar plates.

FIGS. 19A-19B are side views of an implant in a deliverable state and a deployed state, respectively, the implant having plates similar to the implant depicted in FIGS. 2A-2B, but the implant 610 employing one or more helical springs in place of the jack employed in the implant of FIGS. 2A-2B.

FIGS. 20A-20B are the same views as FIGS. 19A-19B, except the implant employs a leaf spring.

FIG. 21A is an elevation view of a fractured vertebral body with rods inserted therein.

FIG. 21B is an isometric view of a jack for use with the rods.

FIG. 21C is the same view as FIG. 21A, except the jack has been attached to the rods and deployed to drive the rods and reduce the fracture.

FIG. 21D is the same view as FIG. 21C, except in cross section, the jack being located within the volume of the vertebral body.

### DETAILED DESCRIPTION

Disclosed herein is an intramedullary implant 10 for reduction of vertebral fractures. In some embodiments, the implant 10 is delivered minimally invasively (e.g., percutaneously) as a whole or complete device into the intramedullary volume of a vertebral body. In other embodiments, the implant 10 is minimally invasively delivered partially or completely disassembled and then assembled in the intramedullary volume of the vertebral body. In yet other embodiments, the implant 10 includes a plurality of incremental components that are incrementally delivered minimally invasively into the intramedullary volume of the vertebral body. The in vivo accumulation or build up of the incremental components forms the implant 10 and acts to reduce the fracture. In any of these embodiments, the implant 10 acts against the interior bone of the intramedullary volume of the vertebral body to reduce the vertebral body generally to its pre-injury configuration.

The implant 10 and its delivery systems and methods are advantageous for a number of reasons. First, the minimally invasive techniques employed to deliver the implant minimizes patient discomfort and soft tissue damage. As the implant 10 is intramedullary, it is a load-sharing, not a load-shielding device, thereby promoting the remodeling performance of the bone after the fracture heals and leading to better long-term strength of the bone tissue. Also, as the implant 10 is intramedullary, the implant 10 does not result in irritation to the soft tissue, and revision surgeries are less likely to be needed. Due in part to its various configurations, the implant 10 is highly adaptable to a wide variety of vertebral fractures with respect to location and severity. The configuration, deployment and minimally invasive characteristics of the implant 10 may reduce by approximately 50% the number of surgical steps needed to repair a vertebral fracture as compared to repairing the same vertebral fracture with plates, cages or other devices known in the art, saving surgical time, costs and risks to the patient from lengthened procedure times. Finally, because the implant 10 in its various embodiments positively maintains the vertebral body in the reduced configuration and remains in the vertebral body, the vertebral body remains in the reduced configuration throughout the surgical procedure and during the subsequent healing period, providing superior results to those found in the art.

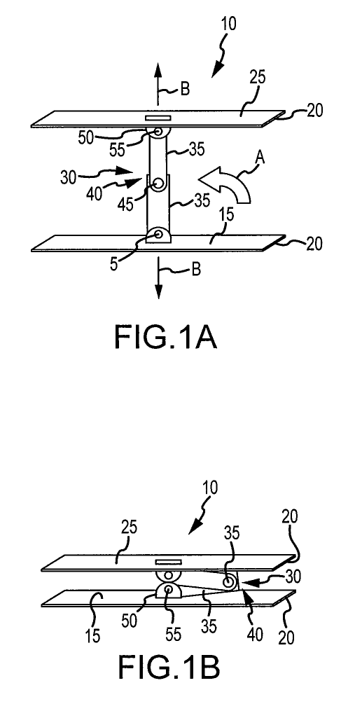

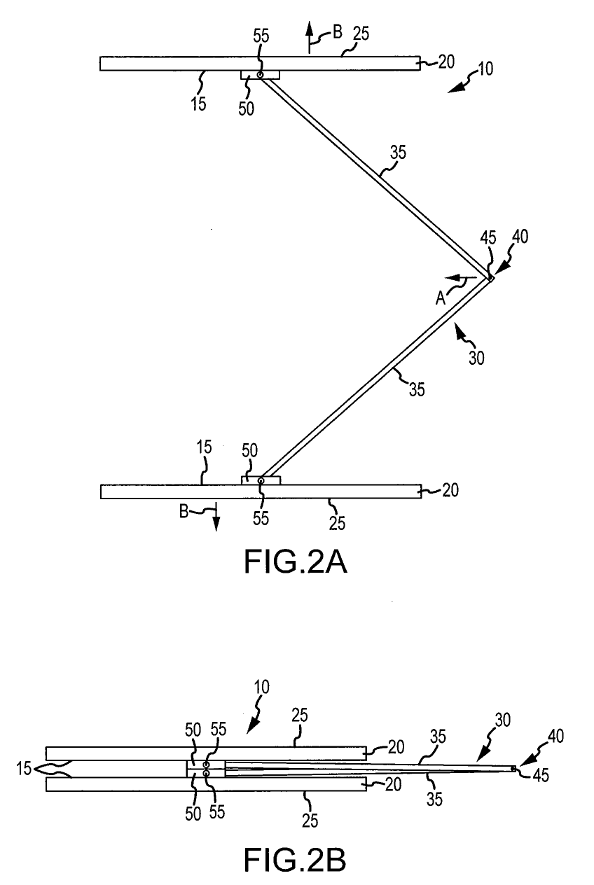

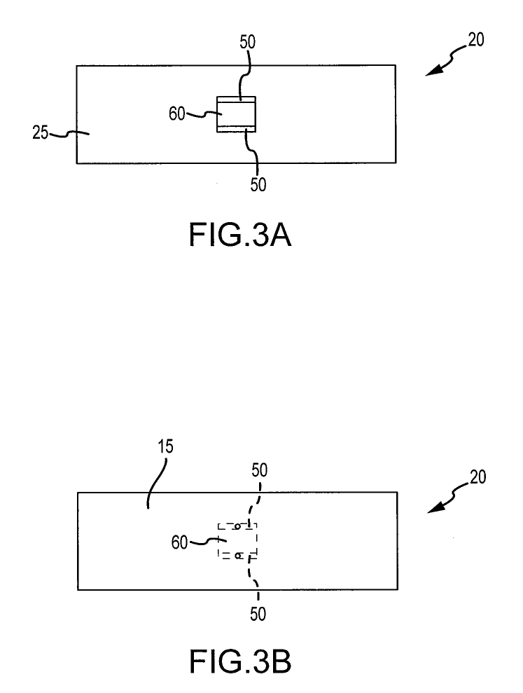

For a detailed discussion of an embodiment of the vertebral implant 10 disclosed herein, reference is made to FIGS. 1A-3B. FIGS. 1A and 1B are side isometric views of the implant in a deployed state and in a collapsed or delivery state, respectively. FIGS. 2A ad 2B are side views of an implant similar to the implant depicted in FIGS. 1A and 1B, the implant depicted in a deployed state and in a collapsed or delivery state, respectively. FIGS. 3A and 3B are plan views of the coupling side 15 of a plate 20 of the implant 10 and a bone engagement side 25 of the plate 20, respectively.

As shown in FIGS. 1A-2B, in one embodiment, the implant 10 includes a first plate 20 and a second plate 20 joined to each other via a linkage or jack assembly 30. In one embodiment, the linkage includes two or more arms 35, the arms forming an elbow 40 with a pivot pin 45. The ends of the arms 35 opposite the elbow 40 are each pivotally coupled to flanges 50 via pivot pins 55.

As indicated in FIGS. 3A-3B, in one embodiment, the flanges 50 are defined from the plate 20, itself. For example, the flanges 50 may be formed in a plate 20 via punching a hole 60 in the center of the plate 20. Depending on the embodiment, the plates 20 may be between approximately 0.5 cm and approximately 3.0 cm long, between approximately 0.2 cm and approximately 2.0 cm wide, and between approximately 0.01 cm and approximately 0.5 cm thick. The plates 20 may be formed from a variety of metals, polymers, or shape memory materials, such as, for example, Nitinol. The legs and other components of the implant may be formed of biocompatible metals (e.g., stainless steel, etc.) or polymers.

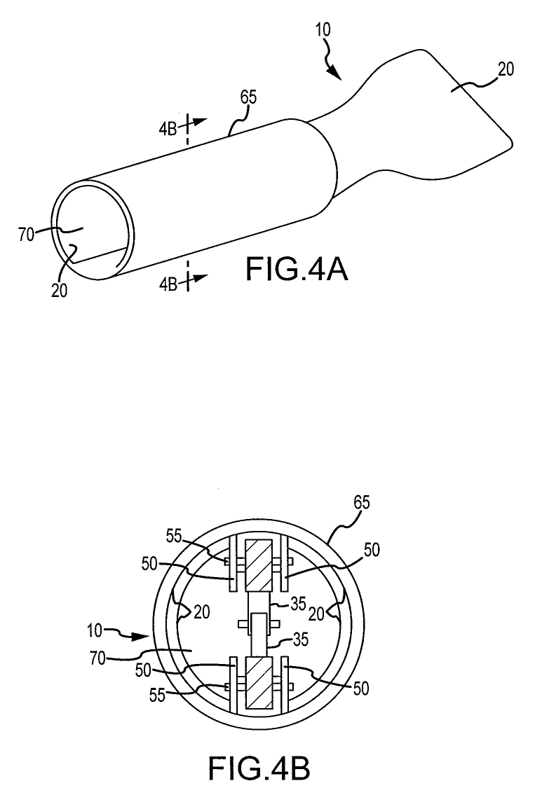

As can be understood from FIGS. 4A and 4B, which are, respectively, a side isometric view of the implant 10 exiting a delivery tubular body (e.g., trocar, introducer sheath, needle, etc.) 65 and a transverse cross sectional view of the tubular body 65 and implant 10 as taken along section line 4B-4B in FIG. 4A, the plates 20 can be formed of a pliable shape memory material or other resilient material so as to allow the plates 20 to be rolled up about each other when the implant 10 is in the delivery state depicted in FIGS. 2A and 2B. As indicated in FIG. 4A, due to being formed of a shape memory material or other resilient material, when the plates begin to be free of the confines of the lumen 70 of the delivery tubular body 65 as the plates exist the distal end of the delivery tubular body 65, the plates 20 bias back into their generally planar configuration depicted in FIGS. 1A, 2A and 3A-3B.

As illustrated in FIGS. 1A and 2A, causing the elbows 40 to move inward as indicated by arrows A causes the plates 20 to move away from each other as indicated by arrows B. Accordingly, as the implant is so deployed, the plates 20 may act against bone to reduce a vertebral fracture, as described below in detail.

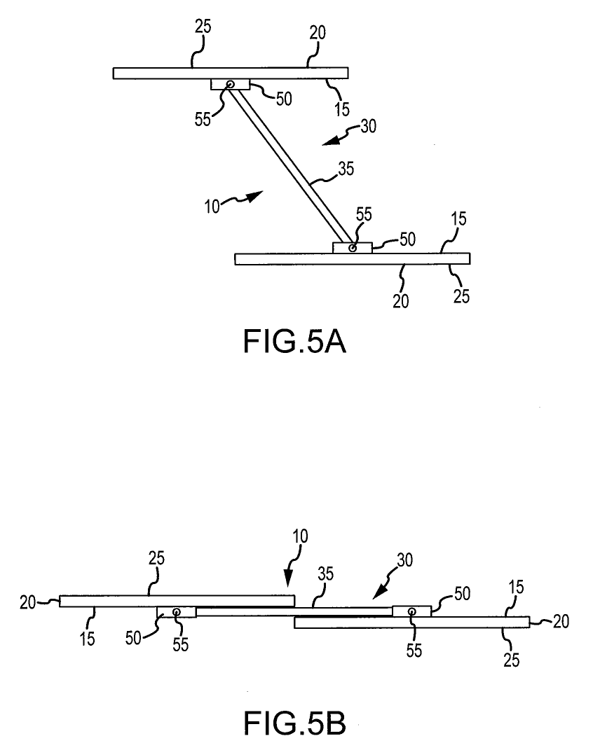

As shown in FIGS. 5A and 5B, which are side views of the implant in a deployed state and in a collapsed or delivery state, respectively, the implant 10 may have a single arm 35 for the jack 30 and, consequently, no elbow 40. Each end of the arm 35 for the jack 30 is pivotally coupled to the flanges 50 of the respective plates 20 via pivot pins 55. In other embodiments, the jack 30 may a scissors arrangement. The jack 30 may have parallel sets of arms (in either the single arm, double arm or scissors configurations).

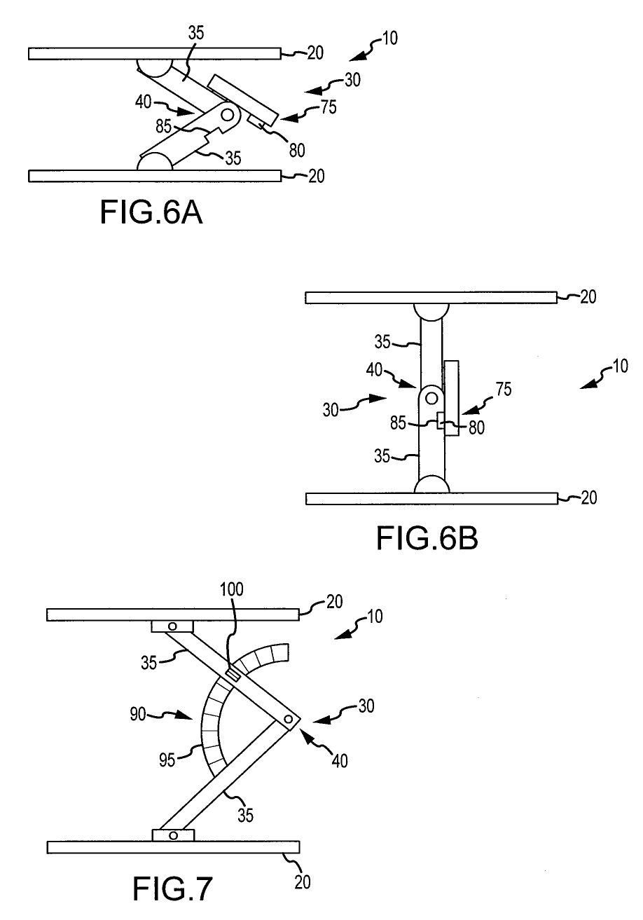

As illustrated in FIGS. 6A-6B, which are side views of the implant in a partially deployed state and a maximum deployed state, respectively, the jack 30 may be equipped with a lock arrangement 75 that maintains the jack 30, or, in other words, the implant 10, in the maximum deployed state. For example, the lock arrangement 75 may include a male latch member 80 supported off of one of the legs 35 that engages a female latch member 85 supported off of the other of the legs 35, thereby locking the implant 10 in the fully deployed state.

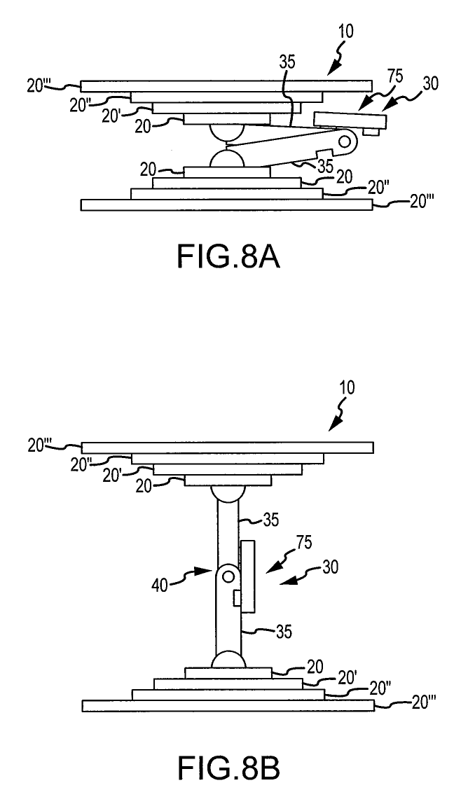

As indicated in FIG. 7, which is a side view of the implant partially deployed, the jack 30 includes a ratchet assembly 90. For example, a ratchet surface 95 is supported off of one of the legs 35 and a pawl tooth 100 supported off of the other of the legs 35. As the jack 30 is increasingly deployed such that the elbow becomes increasingly obtuse with respect to its angle of bend, the pawl tooth 100 will ratchet along the ratchet surface 95. As a result, the ratchet assembly 90 will maintain in the maximum extent of deployment yet reached.

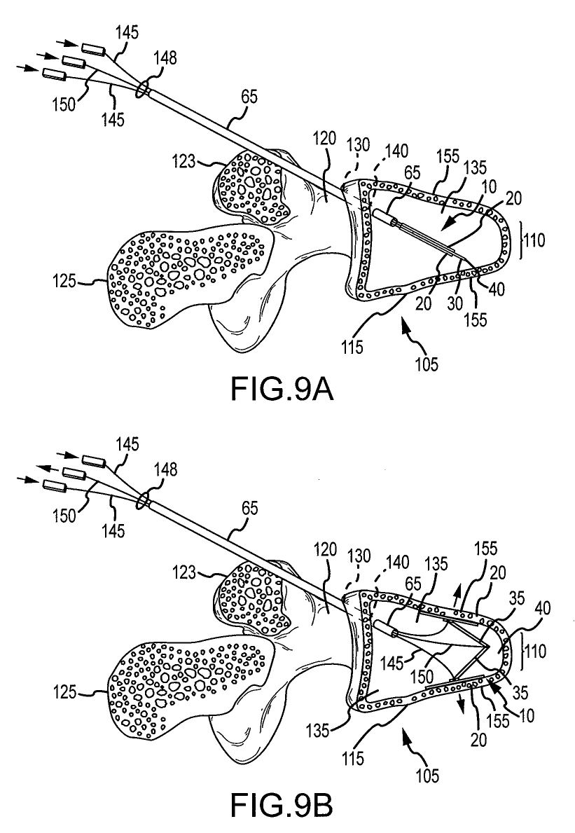

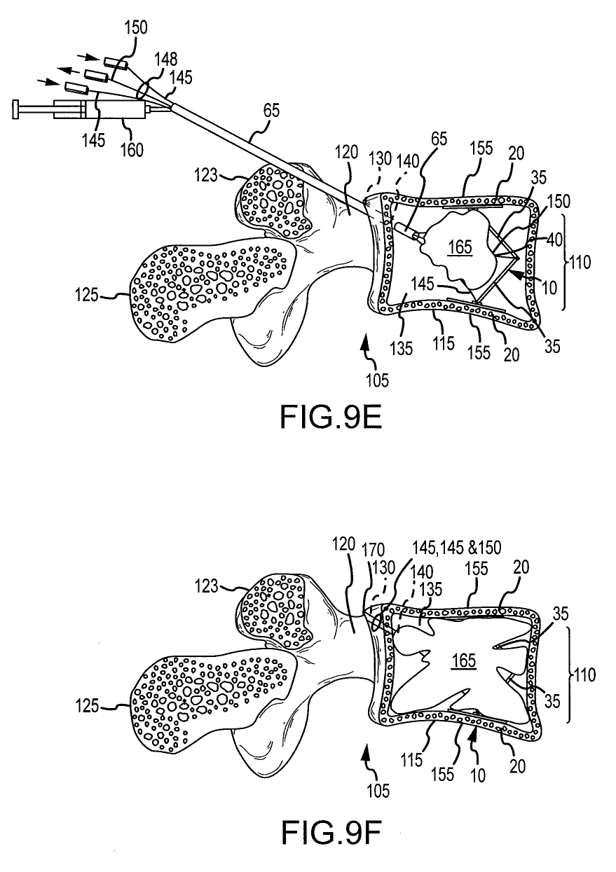

As indicated in FIGS. 8A and 8B, which are side isometric views of the implant in a collapsed or delivery state and a deployed state, respectively, additional plates 20′, 20″, 20″ may be delivered to and stacked on top of the plates 20 of the implant 10 to increase the rigidity of the plates 20. Further description of this embodiment is provided below with respect to FIGS. 10A-10B.

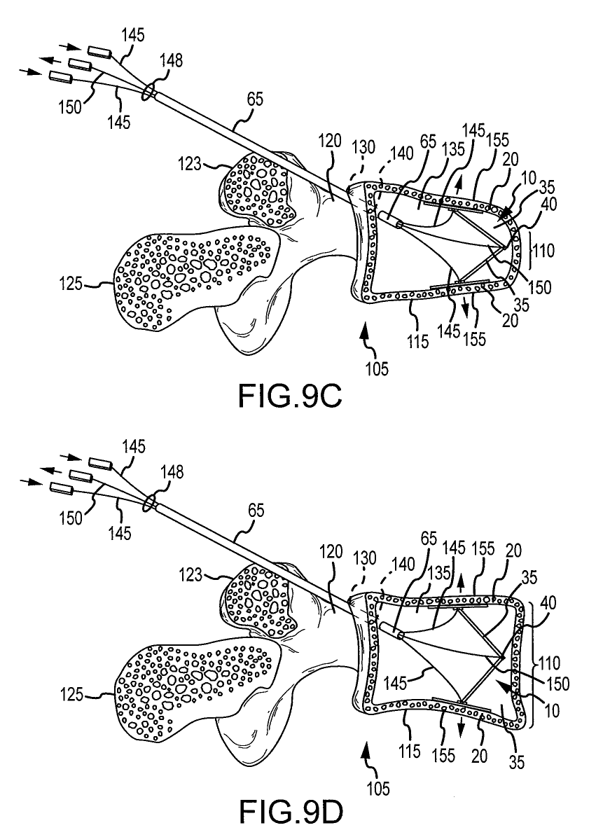

For a discussion of a method of minimally invasively delivering the implant 10 depicted in FIGS. 2A-2B, reference is made to FIGS. 9A-9F, which are posterior-anterior cross sectional elevations of a vertebra 105 having a fracture 110 at a anterior portion of the vertebral body 115, wherein FIGS. 9A-9F depict the progression of the process of reducing the fracture with the implant 10. As can be understood from FIGS. 9A-9F, in addition to the vertebral body 115 and the fracture 110 at the anterior portion of the vertebral body, the vertebra 105 also includes a pedicle 120, a transverse process 123 and a spinous process 125.

As indicated in FIG. 9A, a trocar or cannula 65 extends into the pedicle 120 at a penetration location 130 and extends into the volume or intramedullary space 135 of the vertebral body 115 via another location 140, the trocar staying entirely within bone from the pedicle penetration location 130 to the distal end of the trocar. While the embodiment depicted in FIGS. 9A-9F illustrates the trocar 65 penetrating the pedicle 120 at a location 130 near the top surface of the pedicle 120 and being inclined and passing laterally to the side of the transverse process 123, in other embodiments, the trocar 65 may extend in a more horizontal fashion into the pedicle 120 beginning, for example, at a location near the base of the transverse process and extending the length of the pedicle into the volume of the vertebral body. In other embodiments, other approaches and paths are used to access the volume of the vertebral body.

As shown in FIG. 9A, a pushing device 148 is used to force the implant 10 in the collapsed or delivery state (see FIG. 2B) distally through the trocar 65 until the implant 10 is delivered into the volume 135 of the vertebral body 115. In one embodiment, the pushing device 148 may be a push rod, sheath or other tubular member for extending down the lumen of the trocar to push against the proximal end of the implant, causing the implant to travel distally down the lumen of the trocar to the volume of the vertebral body. In other embodiments as depicted in FIG. 9A, the pushing device 148 may be configured to be a push-pull device 148 having push members 145 and a pull member 150. As best understood from FIGS. 9B-9C, in one embodiment, the distal end of the push members 145 are coupled to the implant 10 near the pivot pin location between the plates 20 and legs 35, and the distal end of the pull member 150 is coupled to the implant near the elbow pivot pin location 40 between the legs 35.

As indicated in FIGS. 9A-9D, once the implant is delivered into the volume 135 of the vertebral body 115, the push members 145 are progressively pushed (as indicated by the associated arrows) as the pull member 150 is progressively pulled (as indicated by the associated arrows). As a result, the plates 20 of the implant 10 are increasingly displaced away from each other, thereby causing the superior and inferior end plates 155 of the vertebral body 115 to displace away from each other. Specifically, the displacement away from each other of the plates 20 may compress the cancellous bone of the volume of the vertebral body 115 until the force of the displacing plates 20 is transferred to the cortical bone forming the superior and inferior end plates 155 of the vertebral body 115, thereby causing the end plates 115 to displace away from each other and fully reducing the fracture 110 of the vertebral body 115 as indicated in FIG. 9D.

As shown in FIG. 9E, in one embodiment, a syringe 160 or similar device is used to inject into the volume 135 of the vertebral body 115 PMMA bone cement 165, PLA-PGA, hydrogel, bone morphogenetic protein, stem cells, cadaver bone, bioengineered matrix, etc. The cement 165 or other mentioned material is injected into the volume 135 of the vertebral body 115 until the volume 135 is generally filled to the greatest extent possible and the deployed implant 10 is imbedded therein as indicated in FIG. 9F. The cement 165 or other mentioned material may be percutaneously delivered in the form of a liquid, gel, paste, or slurry.

As depicted in FIG. 9F, in one embodiment, the push-pull members 145, 150 are cut off at the pedicle opening 130 and a crimp, button or other member 170 is coupled to the cut off push-pull members 145, 150 to hold the push-pull members in place, thereby causing the implant 10 to remain in the fully deployed state depicted in FIGS. 9D-9F.

In other embodiments, a push-pull members 145, 150 may be temporarily coupled to the appropriate locations on the implant 10 (e.g., via hooks, tabs, etc.) and then completely decoupled from the implant and removed from the patient. In such an embodiment, the locking mechanism 75 of FIGS. 6A and 6B or the ratcheting mechanism 90 of FIG. 7 may be employed to retain the implant in the fully deployed state once the push-pull members are removed from the deployed implant.

In summary, with respect to the embodiments depicted in FIGS. 1A-9F, there are two metal elements that form plates that would be folded into the trocar, attached by a hinge mechanism. With an introducer to push the implant forward into the vertebral body, the implant would then be advanced beyond the cannula and, within the vertebral body, transconfiguring from an open curved shape to a more planar shape. Using a trocar within the catheter, the hinge mechanism would be changed from a more acute angle to a more obtuse angle, thereby increasing the distance between the two plates. This would mechanically improve the bone position by performing a “reduction maneuver.” With locking mechanisms within the hinge, once the satisfactory position had been achieved, the hinge could be locked into position. This could be performed using a ratcheting mechanism, a locking mechanism or a crimping mechanism.

As can be understood from FIGS. 10A and 10B, which are the same views as FIGS. 9A and 9B, except depicting the embodiment of FIGS. 8A and 8B being deployed, a delivery sheath 180 can be routed down the trocar 65 to deliver the supplemental plates 20′, 20″ discussed with respect to FIGS. 8A and 8B. Specifically, the implant 10 is delivered via the trocar 65 as discussed above with respect to FIG. 9A. The delivery sheath 180 is then routed down the trocar until the distal end of the sheath 180 projects from the distal end of the trocar. The supplemental plates 20′, 20″ rolled up into a cylinder are routed down the lumen of the delivery sheath into the volume 135 of the vertebral body 115 by being pushed via a member inserted down the sheath behind each supplemental sheath. Once in the volume 135, the supplemental sheaths are allowed to bias back into their generally planar state depicted in FIGS. 8A and 8B. The supplementary plates are positioned as indicated in FIGS. 10A-10B via the sheath 180 or the push member extending through the sheath. The implant is then deployed and implanted as discussed above with respect to FIGS. 9B-9F.

In summary, as discussed with respect to FIGS. 8A-8B and 10A-10B, a series of plates can be arranged in a stack fashion with the larger plates on the outer surfaces and the successively smaller plates on the inner surfaces closest to the hinge mechanism, offering increased strength or stiffness for the combined plates.

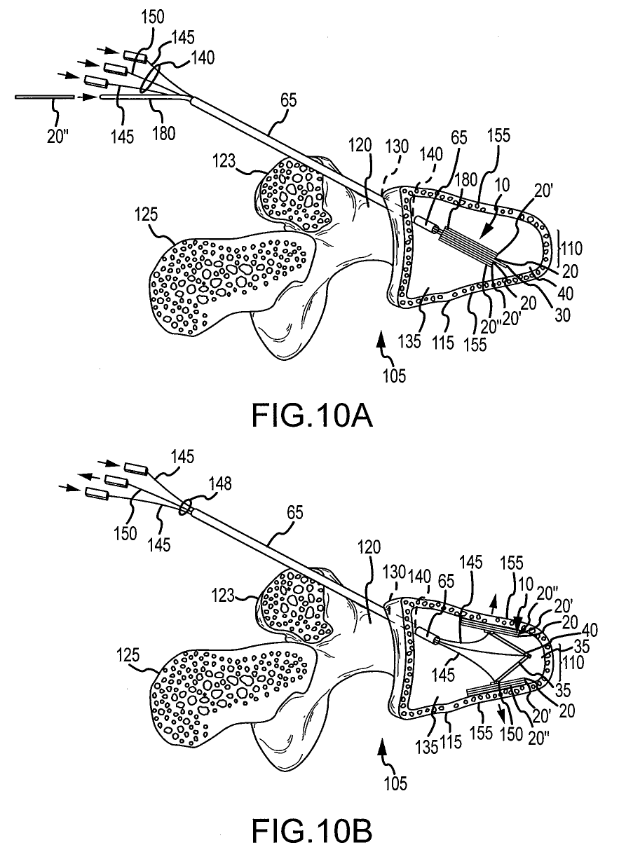

For a discussion of another embodiment, reference is made to FIG. 11, which is a side elevation view (in partial cross section) of a delivery device 200 that incrementally delivers incremental implant elements 205 that accumulate to form the implant 210. As illustrated in FIG. 11, the device 200 includes a handle 215, a trigger 220, a spring 225, a plunger rod 230, a hopper 235, a trocar 240, and a path 245 extending between the hopper and a distal end of the trocar. The trigger is pivotally coupled to the handle and acts against a proximal face of the plunger rod. The plunger rod is biased proximally via the spring and extends into the path 245. The trocar extends distally from the hopper and handle. Pulling the trigger causes the plunger rod to act against the spring and extend down the path. Releasing the trigger allows the spring to bias the plunger proximally. The incremental implant elements 205 fill the hopper 235 and enter the path 245. Biased end lips 250 retain the elements 205 in the inside the trocar path until distal displacement of the plunger rod 230 via trigger actuation causes one or more elements 205 to be ejected past the end lips 250. Depending on the stroke of the plunger rod 230, one, two, three or more elements 205 may be ejected per actuation of the trigger 220.

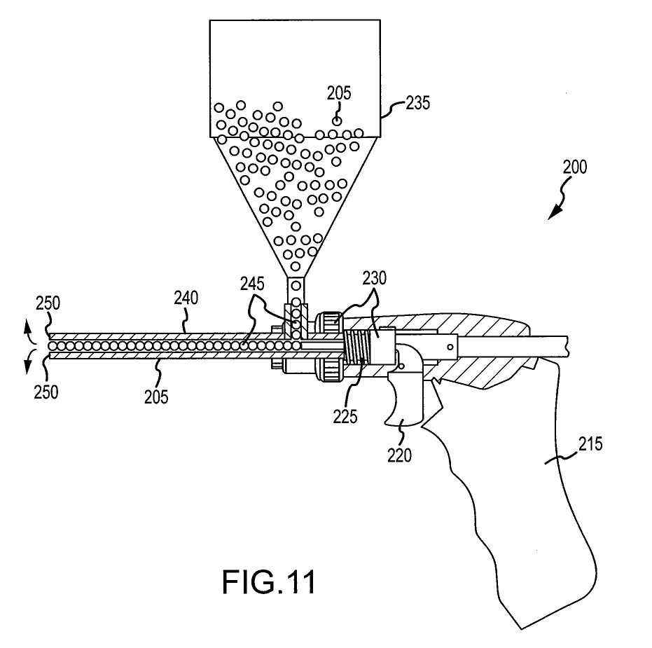

For a discussion of a method of minimally invasively delivering the incremental implant elements 205 to create the accumulated implant 210, reference is made to FIGS. 12A-12E, which are posterior-anterior cross sectional elevations of a vertebra 105 having a fracture 110 at a anterior portion of the vertebral body 115, wherein FIGS. 12A-12E depict the progression of the process of reducing the fracture with the accumulated or built up implant 10. As can be understood from FIGS. 12A-12E, in addition to the vertebral body 115 and the fracture 110 at the anterior portion of the vertebral body, the vertebra 105 also includes a pedicle 120, a transverse process 123 and a spinous process 125.

As indicated in FIG. 12A, a trocar or cannula 65 extends into the pedicle 120 at a penetration location 130 and extends into the volume or intramedullary space 135 of the vertebral body 115 via another location 140, the trocar staying entirely within bone from the pedicle penetration location 130 to the distal end of the trocar. While the embodiment depicted in FIGS. 12A-12E illustrates the trocar 65 penetrating the pedicle 120 at a location 130 near the top surface of the pedicle 120 and being inclined and passing laterally to the side of the transverse process 123, in other embodiments, the cannula or trocar 65 may extend in a more horizontal fashion into the pedicle 120 beginning, for example, at a location near the base of the transverse process and extending the length of the pedicle into the volume of the vertebral body. In other embodiments, other approaches and paths are used to access the volume of the vertebral body.

As shown in FIG. 12A, the device 200 of FIG. 11 is used to incrementally deliver the implant elements 205 into the volume 135 of the vertebral body 115. In one embodiment, the trocar 240 of the device 200 may be inserted down the lumen of the trocar and into the volume of the vertebral body to deliver the implant elements 205. In other embodiments, the outer trocar 65 is not employed, the trocar 240 of the device 200 simply being inserted in place of the trocar 65

As indicated in FIGS. 12A-12D and as discussed above with respect to FIG. 11, by repeatedly squeezing the trigger 220 of the device 200, the device 200 is used to incrementally deliver the incremental implant elements 205 into the volume 135 of the vertebral body 115. Specifically, the reciprocating plunger rod 230 forces the incremental implant elements 205 into the volume 135 of the vertebral body 115. As the incremental implant elements 205 increasingly accumulate or build up in the volume 135 of the vertebral body 115, the accumulating implant elements compress the cancellous bone of the volume of the vertebral body 115 until the force of the accumulating implant elements is transferred to the cortical bone forming the superior and inferior end plates 155 of the vertebral body 115, thereby causing the end plates 115 to displace away from each other and fully reducing the fracture 110 of the vertebral body 115 as indicated in FIG. 12D.

In one embodiment, the incremental implant elements 205 are spherical and have a generally uniform diameter of between approximately 0.5 mm and approximately 1.0 cm. In other embodiments, the implant elements may have other configurations, such as, for example, discoidal, cylindrical, cubical, pyramidal, star shaped, egg shaped, etc. Depending on the embodiment, candidate materials for elements 205 include biocompatible metals (e.g., stainless steel, titanium, etc.), biocompatible polymers (e.g., nylon, PEBAX, etc.), biocompatible ceramics, biocompatible composites, PMMA bone cement, PLA-PGA, hydrogel, bone morphogenetic protein, stem cells, cadaver bone, bioengineered matrix, or etc.

In some embodiments, the elements 205 are all formed from the same material. Specifically, for example, all of the elements 205 may be a biocompatible metal or all of the elements 205 may be PMMA bone cement, PLA-PGA, hydrogel, bone morphogenetic protein, stem cells, cadaver bone, bioengineered matrix, or etc.

In other embodiments, the elements 205 may be formed of multiple materials (e.g., some of the elements 205 are formed from a first material and other elements 205 are formed from a second or third material). Specifically, for example, one quarter to half of the elements 205 may be a biocompatible metal and three quarters to half of the elements 205 may be PMMA bone cement, PLA-PGA, hydrogel, bone morphogenetic protein, stem cells, cadaver bone, bioengineered matrix, or etc. Such varied types of elements 205 may be sequenced within the device 200 to ensure the elements 205 mix as desired in the volume of the vertebral body. For example, the dispenser device 200 could have metal elements 205 and bioactive elements 205 alternating, or first 10 elements 205 metal, followed by a few bioactive elements 205 or bioactive/metal elements 205 (i.e., composite elements 205 discussed next).

In some embodiments, the elements 205 may have composite construction formed of multiple materials (e.g., individual elements 205 are formed from two or more materials). Specifically, for example, the elements 205 may have a biocompatible metal outer porous shell and the interior of the shell is filled with PMMA bone cement, PLA-PGA, hydrogel, bone morphogenetic protein, stem cells, cadaver bone, bioengineered matrix, or etc. Alternatively, for example, the elements 205 may have a biocompatible metal outer inner core and an outer shell formed of PMMA bone cement, PLA-PGA, hydrogel, bone morphogenetic protein, stem cells, cadaver bone, bioengineered matrix, or etc.

In one embodiment as shown in FIG. 12E, once the accumulated implant 210 has fully reduced the fracture 110, the trocar 65 and device 200 may be removed and the penetrations 130, 140 may be sealed. Where some of the elements 205 or aspects of some of the elements includes PMMA bone cement, PLA-PGA, hydrogel, bone morphogenetic protein, stem cells, cadaver bone, bioengineered matrix, or etc., such materials will act to facilitate inducing bone growth into the accumulated implant 210.

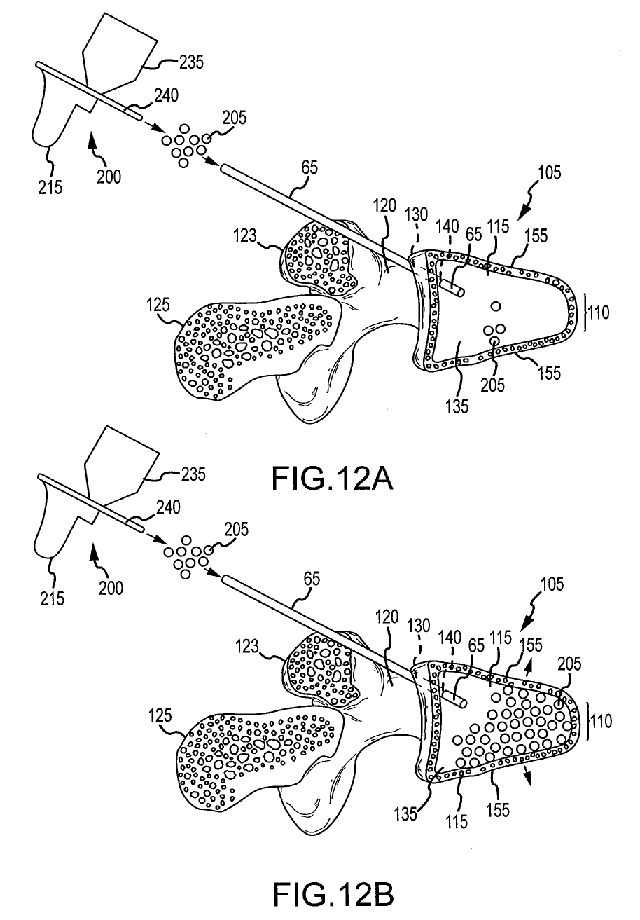

As shown in FIGS. 13A and 13B, which are the same views of the fully reduced vertebral body 115 as FIG. 12E, in one embodiment, a syringe 160 or similar device is used to inject into the volume 135 of the vertebral body 115 PMMA bone cement 165, PLA-PGA, hydrogel, bone morphogenetic protein, stem cells, cadaver bone, bioengineered matrix, etc. The cement 165 or other mentioned material is injected into the volume 135 of the vertebral body 115 until the volume 135 is generally filled to the greatest extent possible and the accumulated implant 210 is imbedded therein as indicated in FIG. 13B.

In some embodiments as can be understood form FIGS. 13A-13B, the elements 205 could be dispersed from the dispenser device 200 mixed in with the above mentioned liquid or paste material 165 in the form of a grout. Thus, there would be one step to achieving the arrangement of elements 205 and liquid or paste material 165 depicted in FIG. 13B.

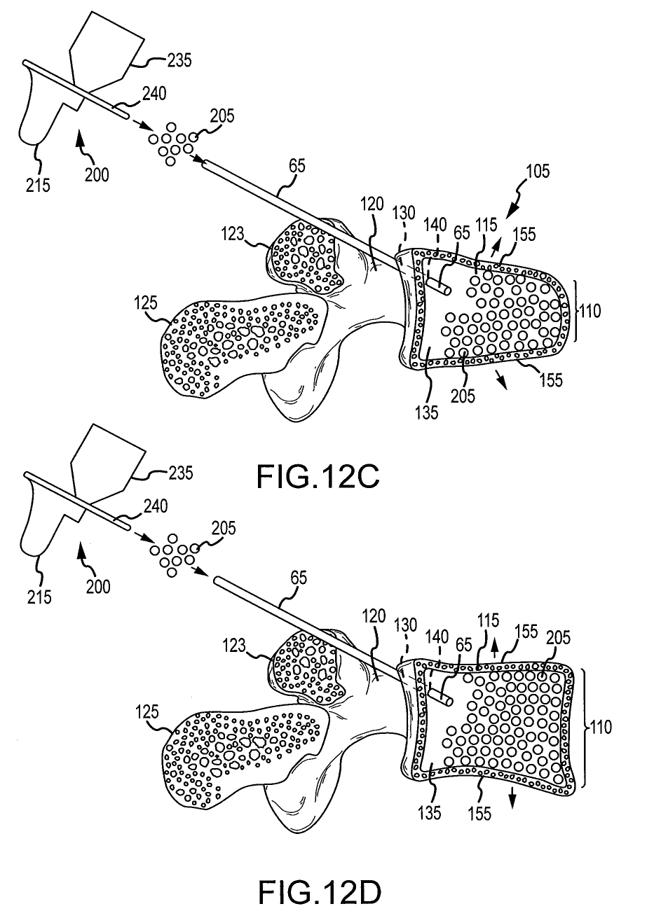

For a discussion of yet another embodiment of an implant element 305 for another accumulated implant 310, reference is made to FIGS. 14A and 14B, which are side view of the implant elements in a constrained state and in a free state, respectively. As shown in FIG. 14A, the helical spring implant element 305 when in a constrained state (e.g., when constrained within a lumen of a trocar or cannula 65), the element 305 is a helical spring having a reduced diameter A and an enlarged length X. As shown in FIG. 14B, the helical spring implant element 305 when in a free unconstrained state (e.g., when free of the lumen of a trocar or cannula 65), the element 305 is a helical spring having a enlarged diameter B and an reduced length Y, wherein X is greater than Y and A is less than B.

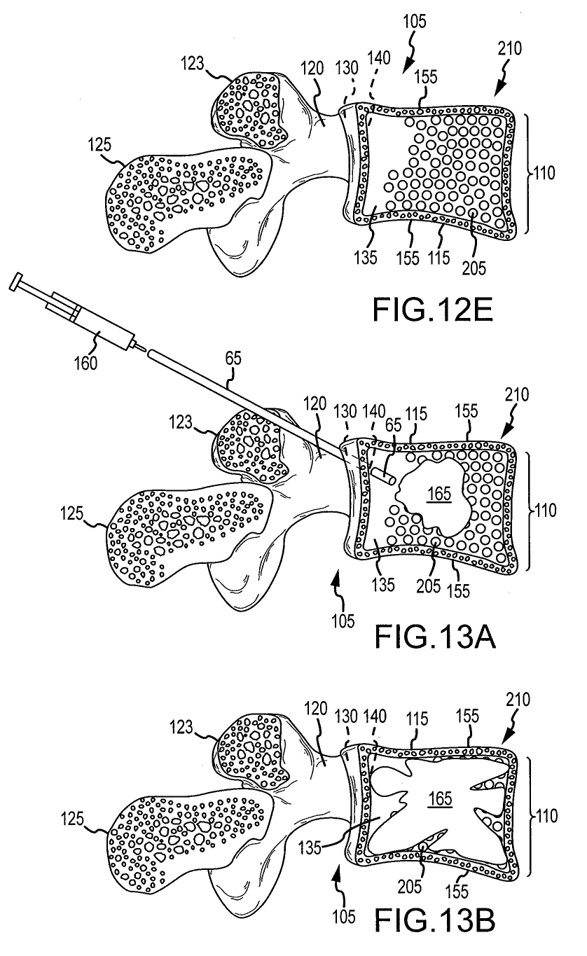

As shown in FIGS. 15A and 15B, which are the same views as FIGS. 12A and 12B, except of the helical spring implant elements of FIGS. 14A and 14B being deployed, a trocar or cannula 65 extends into the pedicle 120 at a penetration location 130 and extends into the volume or intramedullary space 135 of the vertebral body 115 via another location 140, the trocar staying entirely within bone from the pedicle penetration location 130 to the distal end of the trocar. While the embodiment depicted in FIGS. 15A-15B illustrates the trocar 65 penetrating the pedicle 120 at a location 130 near the top surface of the pedicle 120 and being inclined and passing laterally to the side of the transverse process 123, in other embodiments, the cannula or trocar 65 may extend in a more horizontal fashion into the pedicle 120 beginning, for example, at a location near the base of the transverse process and extending the length of the pedicle into the volume of the vertebral body. In other embodiments, other approaches and paths are used to access the volume of the vertebral body.

As shown in FIG. 15A, the a push rod 315 may be used to incrementally deliver the helical spring implant elements 305 into the volume 135 of the vertebral body 115. In doing so, the spring elements 305 are loaded into the proximal end of the trocar 65 in the constrained state (reduced diameter state) depicted in FIG. 14A. The push rod shoves the element 305 down into the volume of the vertebral body. As indicated in FIG. 15A, upon exiting the distal end of the trocar 65 into the volume 135 of the vertebral body 115, the spring element 305 is free to assume the unconstrained state (increased diameter state) illustrated in FIG. 14B. In some embodiments, to facilitate the elements 305 freely biasing into the unconstrained state of FIG. 14B, the rod 315 is first inserted into the volume of the vertebral body via the trocar to compact the cancellous bone and provide a volume in which the spring implant elements 305 may increase in diameter.

The delivery of the elements 305 into the volume of the vertebral body is repeated as indicated in FIG. 15B to result in an accumulated implant 310. In a manner similar to that discussed with respect to FIGS. 12C-12E, repeating the delivery of the spring elements 305 and the biasing of the elements 305 into their enlarged diameters will eventually result in the full reduction of the fracture 110. Specifically, the accumulated implant 310 acts against the cancellous bone which transfers the force to the cortical bone forming the superior and inferior end plates 155 of the vertebral body, thereby reducing the fracture. At this point, the surgery may be completed, or, alternatively, PMMA bone cement, PLA-PGA, hydrogel, bone morphogenetic protein, stem cells, cadaver bone, bioengineered matrix, or etc. may be injected into the volume of the vertebral body in a manner similar to that discussed above with respect to FIGS. 13A and 13B.

In one embodiment, the helical spring implant elements 305 have a constrained diameter A of between approximately 0.5 mm and approximately 5 mm and a constrained length X of between approximately 0.5 cm and approximately 4 cm. The helical spring implant elements 305 have a unconstrained diameter B of between approximately 0.5 cm and approximately 4 cm and a unconstrained length Y of between approximately 1 cm and approximately 4 cm. In one embodiment, the spring implant elements 305 are formed from stainless steel.

In one embodiment of the spring implant element 305 depicted in FIGS. 14A-14B, the spring element 305 is delivered in the reduced diameter configuration depicted in FIG. 14A. Upon being delivered to the volume of the vertebral body, one end of the spring element 305 is fixed, for example, by being screwed, glued, pinned, etc. to bone or being attached to a member at the end of the element 305 that will not rotate within the volume of the vertebral body. The other end of the spring element 305 is then rotated to unwind the spring element until the spring element diameter transitions from diameter A to the desired diameter B, the length also transitioning from length X to length Y. The increased diameter acts to force the vertebral plates 155 away from each other, reducing the fracture. The end of the spring element 305 that is acted on to unwind the element 305 may then be fixed in a manner similar to the end that was already fixed.

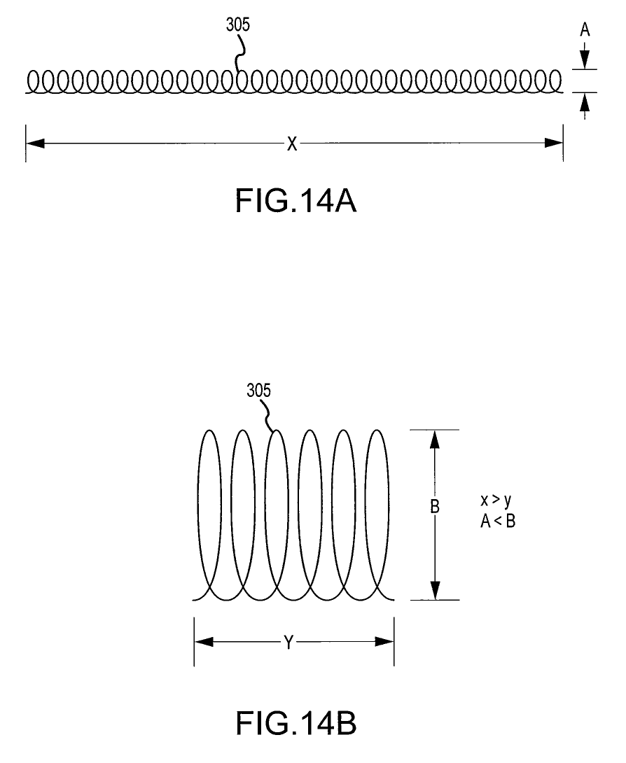

For a discussion of yet another embodiment of an implant element 405 for another accumulated implant 410, reference is made to FIGS. 16A and 16B, which are, respectively, a side view and an end view of the implant elements. As shown in FIGS. 16A and 16B, the incremental elements 405 are cylindrical and have a generally uniform diameter of between approximately 0.1 cm and approximately 2 cm, and a length of between approximately 0.5 cm and approximately 4 cm. Depending on the embodiment, candidate materials for elements 405 include biocompatible metals (e.g., stainless steel, titanium, etc.), biocompatible polymers (e.g., nylon, PEBAX, etc.), biocompatible ceramics, biocompatible composites, PMMA bone cement, PLA-PGA, hydrogel, bone morphogenetic protein, stem cells, cadaver bone, bioengineered matrix, or etc.

In some embodiments, the elements 405 are all formed from the same material. Specifically, for example, all of the elements 405 may be a biocompatible metal or all of the elements 405 may be PMMA bone cement, PLA-PGA, hydrogel, bone morphogenetic protein, stem cells, cadaver bone, bioengineered matrix, or etc.

In other embodiments, the elements 405 may be formed of multiple materials (e.g., some of the elements 405 are formed from a first material and other elements 405 are formed from a second or third material). Specifically, for example, one quarter to half of the elements 405 may be a biocompatible metal and three quarters to half of the elements 405 may be PMMA bone cement, PLA-PGA, hydrogel, bone morphogenetic protein, stem cells, cadaver bone, bioengineered matrix, or etc. The dispenser device 200 of FIG. 11 may be provided with a magazine in place of the hopper 235 that is configured to supply the cylindrical elements 405 in a desired orientation for delivery down the trocar 240. Such varied types of elements 405 may be sequenced within the device 200 to ensure the elements 405 mix as desired in the volume of the vertebral body. For example, the dispenser device 200 could have metal elements 405 and bioactive elements 405 alternating, or first 10 elements 405 metal, followed by a few bioactive elements 405 or bioactive/metal elements 405 (i.e., composite elements 405 discussed next).

In some embodiments, the elements 405 may have composite construction formed of multiple materials (e.g., individual elements 405 are formed from two or more materials). Specifically, for example, the elements 405 may have a biocompatible metal outer porous shell and the interior of the shell is filled with PMMA bone cement, PLA-PGA, hydrogel, bone morphogenetic protein, stem cells, cadaver bone, bioengineered matrix, or etc. Alternatively, for example, the elements 405 may have a biocompatible metal outer inner core and an outer shell formed of PMMA bone cement, PLA-PGA, hydrogel, bone morphogenetic protein, stem cells, cadaver bone, bioengineered matrix, or etc.

In some embodiments, the cylindrical elements 405 of FIGS. 16A-16B are in the form of a solid cylinder that is generally free of voids. In other embodiments, the cylinder elements 405 are tubular (e.g., generally hollow).

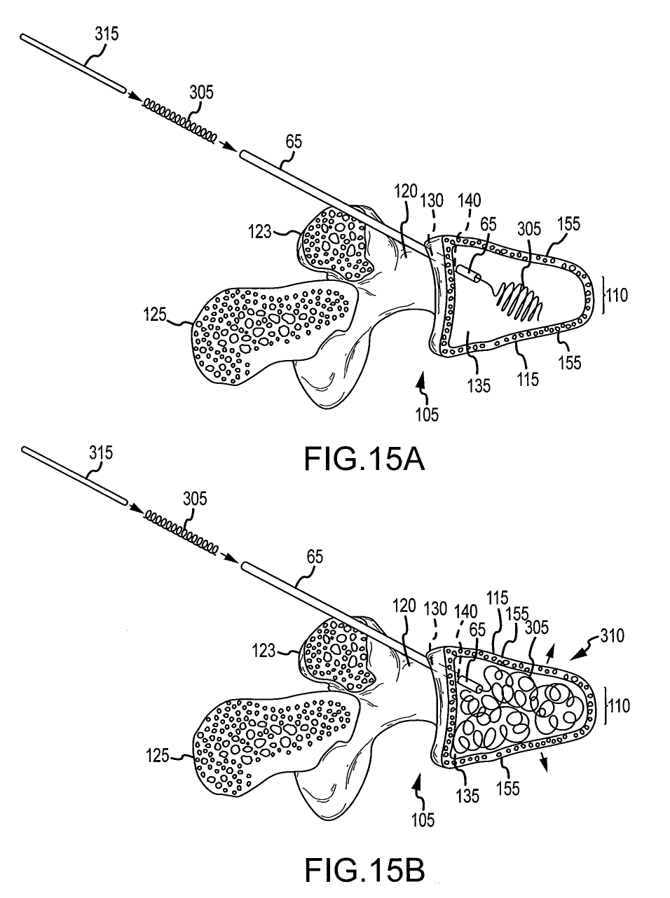

As shown in FIGS. 17A and 17B, which are the same views as FIGS. 12A and 12B, except of the cylindrical implant elements of FIGS. 16A and 16B being deployed, a trocar or cannula 65 extends into the pedicle 120 at a penetration location 130 and extends into the volume or intramedullary space 135 of the vertebral body 115 via another location 140, the trocar staying entirely within bone from the pedicle penetration location 130 to the distal end of the trocar. While the embodiment depicted in FIGS. 17A-17B illustrates the trocar 65 penetrating the pedicle 120 at a location 130 near the top surface of the pedicle 120 and being inclined and passing laterally to the side of the transverse process 123, in other embodiments, the cannula or trocar 65 may extend in a more horizontal fashion into the pedicle 120 beginning, for example, at a location near the base of the transverse process and extending the length of the pedicle into the volume of the vertebral body. In other embodiments, other approaches and paths are used to access the volume of the vertebral body.

As shown in FIG. 17A, the a push rod 315 or a modified version of the device 200 of FIG. 11 may be used to incrementally deliver the cylindrical implant elements 405 into the volume 135 of the vertebral body 115. In doing so, the elements 405 are loaded into the proximal end of the trocar 65 and the push rod shoves the element 405 down into the volume of the vertebral body.

The delivery of the elements 405 into the volume of the vertebral body is repeated as indicated in FIG. 17B to result in an accumulated implant 410. In a manner similar to that discussed with respect to FIGS. 12C-12E, repeating the delivery of the elements 405 into the volume of the vertebral body will eventually result in the full reduction of the fracture 110. Specifically, the accumulated implant 410 acts against the cancellous bone which transfers the force to the cortical bone forming the superior and inferior end plates 155 of the vertebral body, thereby reducing the fracture. At this point, the surgery may be completed, or, alternatively, PMMA bone cement, PLA-PGA, hydrogel, bone morphogenetic protein, stem cells, cadaver bone, bioengineered matrix, or etc. may be injected into the volume of the vertebral body in a manner similar to that discussed above with respect to FIGS. 13A and 13B.

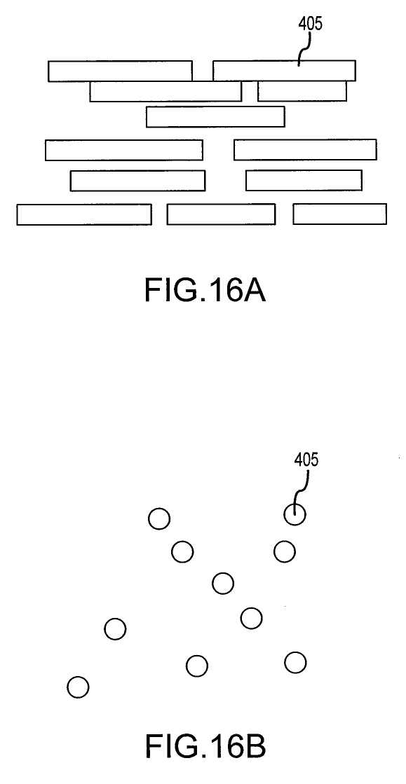

In one embodiment, the cylinder elements 405 of FIGS. 16A-16B are plates similar to the plates 20 of FIGS. 1A and 1B that are delivered in a rolled up cylindrical state (as depicted in FIGS. 4A-4B), but bias into a planar state once exiting the trocar 65. As can be understood from FIG. 18, which is a side view of an accumulated implant 510 employing cylinder elements 405 that has transconfigured into planar plates similar to those described with respect to FIGS. 1A, 1B, 4A and 4B, additional implant elements 505 may be delivered into the volume of the vertebral body. The additional elements 505 may be shaped to have orientation features or engagement features. For example, the elements 505 may be shaped or configured to mate or interlock similar to LEGOS®, allowing the elements 405 and 505 to be assembled within the volume of the vertebral body to build the accumulated or built up implant 510.

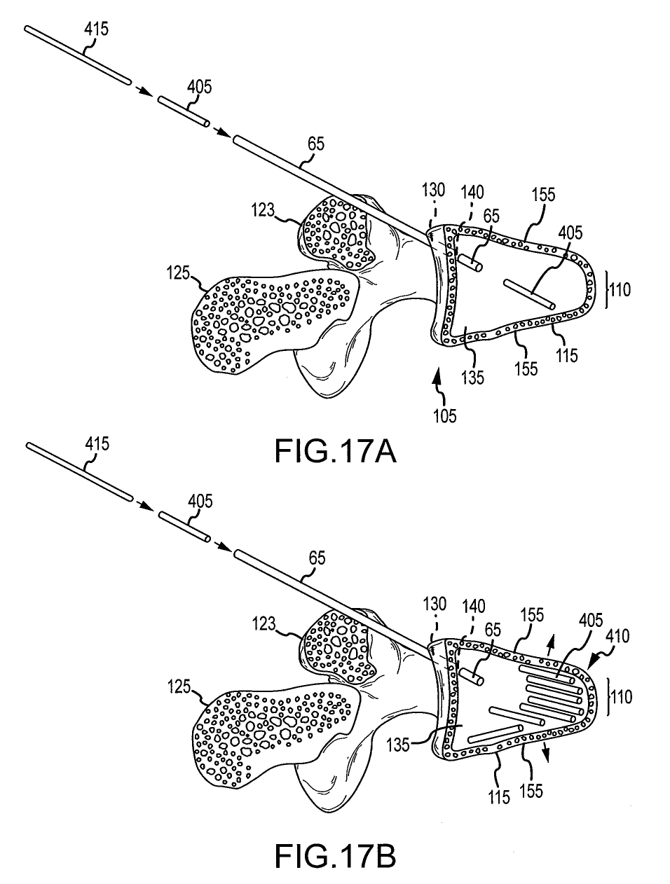

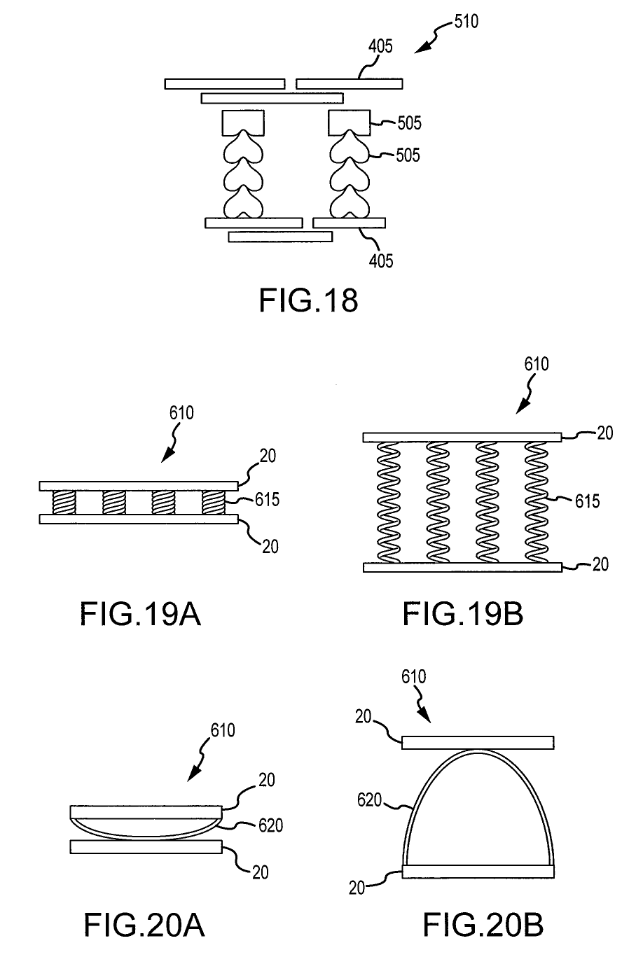

In some embodiments, the implant 20 of FIGS. 2A-2B may have a mechanism for deployment that is generally automatic. For example, as shown in FIGS. 19A-19B, which are side views of an implant 610 in a deliverable state and a deployed state, respectively, the implant 610 has plates 20 similar to the implant 10 depicted in FIGS. 2A-2B, but the implant 610 employs one or more helical springs 615 in place of the jack 30 employed in the implant of FIGS. 2A-2B. In other embodiments, as depicted in FIGS. 20A-20B, which are the same views as FIGS. 19A-19B, the implant 610 may employ a leaf spring 620 instead of the helical springs 615.

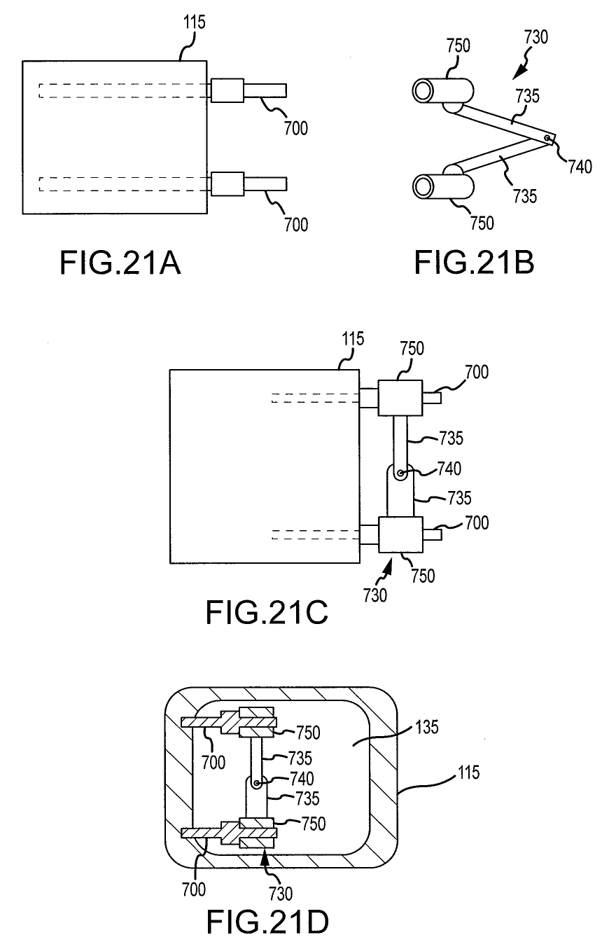

As can be understood from FIG. 21A, which is an elevation view of a fractured vertebral body 115, rods 700 may be inserted into the fractured vertebral body 115. The rods 700 project from the vertebral body in a manner that allows the rods 700 to be coupled to a jack 730, which is depicted in the isometric view in FIG. 21B. As shown in FIG. 21B, the jack 730 includes arms 735, a hinged elbow 740 and two couplers 750 for coupling to the exposed ends of the rods 700. One of the ends of each of the arms 735 are pivotally coupled to each other to form the elbow 740. The other ends of the arms 735 are pivotally coupled to the couplers 750. As shown in FIG. 21C, which is the same view as FIG. 21A, except the jack 730 has been attached to the rods 700, the jack 730 can be deployed to drive the rods 700 apart and reduce the fracture. Once deployed as desired, the jack 730 can be locked in place via crimping, locking arrangements, ratchet arrangements, etc.

While the embodiment depicted in FIGS. 21A and 21C illustrates the rods protruding out of the bone and the jack being located on the outside of the bone, in other embodiments, the embodiment may be as depicted in FIG. 21D, which is the same view as FIG. 21C, except in cross section. Specifically, the rods 700 may extend from the cortical bone into the volume 135 of the vertebral body 115, and the jack 730 is located in the volume 135 and coupled to the rods 700 to force the rods apart and reduce the fracture.

In one embodiment, a series of the implants 10 discussed with respect to FIGS. 1A-10B may be sequentially introduced or simultaneously introduced until the arrangement of implants 10 fill the volume of the fractured vertebral body and reduce the fracture.

For any of the embodiments described above wherein the implant is an accumulated implant formed of incremental implant elements delivered via a delivery device (e.g., see FIGS. 11, 12A-12E, and 17A-17B), the delivery device and incremental implant elements may form a system for treating a vertebral fracture and may be provided in the form of a medical kit including the delivery device and the incremental implant elements contained in sterile packaging. Instructions for the kit and system may be available via the packaging or via the internet.

For any of the embodiments discussed above, the cannula, catheter, sheath, or trocar 65 is placed within the patient under fluoroscopic visualization. In one embodiment, the trocar 65 is passed through the pedicle posterolateral to the fractured vertebral body, the tip of the trocar being located inside the posterior aspect of the vertebral body. Any of the above described implants or elements thereof may be provided with features that facilitate the tracking of implant or elements thereof via fluoroscopy. For example, the implants or elements thereof may have distinct shapes on one end that can be seen via fluoroscopy and that indicate the orientation of the implant or elements thereof. The implants or elements thereof may be equipped with radiopaque markers (e.g., tungsten, platinum, etc.) on one end that can be seen via fluoroscopy and that indicate the orientation of the implant or elements thereof.

After the reduction has been performed by one of the implants discussed above, in some embodiments, the introduction trocar can be used to facilitate placement of a catheter through which bone filler, bone void, polymethylmethacrylate bone cement, bone morphogenetic protein substance, or bone graft substance or combination thereof can be introduced into the fractured vertebral body to further stabilize fracture and hold its shape to prevent loss of vertebral body height through compression.

Although the present invention has been described with reference to preferred embodiments, persons skilled in the art will recognize that changes may be made in form and detail without departing from the spirit and scope of the invention.

What is claimed is:

1. A method of reducing a fracture in a vertebra body, the method comprising:
percutaneously introducing an implant into a volume of the vertebra body;
causing first and second members of the implant to displace away from each other within the volume, thereby placing the fracture in the vertebra body in a reduced state; and
fixing the first and second members relative to each other so as to maintain the fracture of the vertebra body in the reduced state.

2. The method of claim 1, wherein causing the first and second members to displace away from each other includes deploying a jack between the members.

3. The method of claim 1, wherein causing the first and second members to displace away from each other includes allowing a spring to bias between the members.

4. The method of claim 1, wherein causing the first and second members to displace away from each other includes accumulating incremental members between the members.

5. The method of claim 1, further comprising percutaneously adding a supplemental member to the at least one of the members.

6. The method of claim 1, further comprising embedding the implant in the volume of the vertebra body via a percutaneously delivered liquid, paste, gel or slurry.

7. The method of claim 1, wherein percutaneously introducing an implant into a volume of the vertebra body includes allowing the members to bias from a rolled state to an unrolled, generally planar configuration.

8. A method of reducing a fracture in a vertebra body, the method comprising:
percutaneously introducing incremental implant elements into a volume of the vertebra body, the accumulation of the incremental elements in the volume of the vertebra body placing the fracture in the vertebra body in a reduced state.

9. The method of claim 8, wherein the incremental implant elements are generally spherical.

10. The method of claim 8, wherein the incremental implant elements are generally cylindrical.

11. The method of claim 8, wherein the incremental implant elements include a first type of incremental implant member and a second type of incremental implant member different from the first type.

12. The method of claim 11, wherein the first and second types are staged relative to each other in a device used to percutaneously introduce the incremental implant elements into the volume of the vertebra.

13. The method of claim 11, wherein the first type includes at least one of a polymer or metal, and the second type includes at least one of a bone growth inducing material.

14. The method of claim 8, further comprising embedding the incremental implant elements in the volume of the vertebra body via a percutaneously delivered liquid, paste, gel or slurry.

15. A system for reducing a fracture in an vertebral body, the system comprising:
incremental implant elements configured for percutaneous delivery into, and accumulation within, a volume of the vertebral body; and
a delivery device configured to percutaneously deliver the incremental implant elements into the volume of the vertebra body in an incremental fashion.

16. The system of claim 15, wherein the delivery device includes a tubular body and a trigger operated mechanism that causes an incremental amount of the incremental implant elements to exit the tubular body with each actuation of the trigger operated mechanism.

17. The system of claim 16, wherein the tubular body includes a trocar, catheter or cannula.

18. The system of claim 16, wherein the trigger operated mechanism includes a trigger coupled to a spring biased plunger rod.

19. The system of claim 15, wherein the incremental implant elements are generally spherical or generally cylindrical.

20. The system of claim 19, wherein the incremental implant elements include a first type of incremental implant element and a second type of incremental implant element different from the first type.

21. The system of claim 20, wherein the first type includes at least one of a polymer or metal, and the second type includes at least one of a bone growth inducing material.

22. The system of claim 15, wherein at least some of the incremental implant elements have a composite configuration.

23. The system of claim 22, wherein outer surfaces of the at least some of the incremental implant elements include a polymer or metal shell, and inner portions of the at least some of the incremental implant elements include a bone growth inducing material.

24. The system of claim 22, wherein outer surfaces of the at least some of the incremental implant elements include a bone growth inducing material, and inner portions of the at least some of the incremental implant elements include polymer or metal core.

25. An intramedullary implant for reducing a fracture in an vertebral body, the implant comprising: generally opposed plates coupled together and configured to deploy from a percutaneously deliverable configuration to a deployed configuration wherein the plates are displaced from each other.

26. The implant of claim 25, wherein the plates are pliable and rolled up about each other when in the percutaneously deliverable configuration.

27. The implant of claim 26, wherein the plates are generally planar when in the deployed configuration.

28. The implant of claim 25, further comprising a jack that assists in displacing the plates from each other.

29. The implant of claim 25, further comprising a spring that assists in displacing the plates from each other.
